(19) United States
(12) Patent Application Publication
(10) Pub. No.: US 2006/0141555 A1
(43) Pub. Date: Jun. 29, 2006

(54) Control of biocatalysis reactions

(75) Inventors: Christopher John Knowles, Oxfordshire (GB); Simon Andrew Jackman, Oxfordshire (GB); Li Hong, Oxfordshire (GB); Robert Mustacchi, Copenhagen (DK); John Garry Sunderland, Chester (GB)

Correspondence Address: B Todd Patterson;Moser Patterson & Sheridan, Suite 1500, 3040 Post Oak Boulevard, Houston, TX, 77056, US

(73) Assignee: C-Tech Innovation Limited, Chester (GB)

(21) Appl. No.: 10/535,687

(22) PCT Filed: Nov. 19, 2003
(86) PCT No.: PCT/GB03/05031

§ 371 Date: May 19, 2005

(30) Foreign Application Priority Data
Nov. 19, 2002 (GB) 0227021.3

(51) Int. Cl. C12P 1/00; C12Q 1/00

(52) U.S. Cl. 435/41; 205/777.5

(57) ABSTRACT

A process for increasing the rate of biocatalysis reactions, which comprises applying a direct current electric field to a reaction mixture, wherein the reaction mixture and the electrodes used to apply said electric field are separated such that the reaction mixture does not come into contact with said electrodes.

The present invention is concerned with controlling biocatalysis reactions, and particularly with controlling the rate of such reactions so as to optimise the yield of bioproducts derived therefrom.

Bioconversion processes are becoming increasingly important to the manufacture of high added value chemical intermediates such as pharmaceuticals, flavours & fragrances, and the like. As a result, bioconversion processes are increasingly being used to replace traditional fine chemicals manufacturing techniques which often require the use of organic solvents and the addition of chemical reagents, which create toxic waste streams requiring expensive treatment to protect the environment. Such chemical processes are also hazardous and energy intensive, since they are operated at high temperatures and pressures.

Bioprocesses also offer extensive possibilities for synthetic routes to organic compounds, which are often difficult to prepare by established chemical methods. The application of biological systems to chemicals manufacturing offers several advantages: high selectivity, with enzymes distinguishing between enantiomers and regio-isomers; use of aqueous reaction media and operation under near-ambient conditions.

Biocatalysis, therefore, offers considerable advantages over traditional processing methods being an intrinsically clean technology which avoids the necessity to add significant quantities of toxic chemicals, while being carried out under ambient pressures and low temperatures.

An important generic factor to be considered during process design and operation is process intensification. High throughput rates are important for cost reduction in terms of capital utilisation and operational costs. Achieving increased reaction rates in biocatalytic processes is therefore a desirable goal.

The rates of biocatalytic processes are limited by numerous factors. For example, the relevant enzymes in the vast majority of whole cell biocatalysis reactions are retained within the cells. Reactions will only occur at a significant rate if the organic chemical substrate has good access to the enzyme complement of the cell, and if the product can be easily recovered from the cell. Developing new methods, which are able to overcome the factors limiting reaction rates, could result in significant potential economic benefits to manufacturing industry.

Biocatalysis therefore offers considerable advantages over traditional processing methods being an intrinsically clean technology, which avoids the necessity to add significant quantities of toxic chemicals, while being carried out under ambient pressures and at low temperatures.

Electroporation has been studied previously in the field of the interactions between electrical fields and living cells. The technique is usually associated with reversible cell membrane permeabilisation, resulting from the application of high voltage electrical fields (about 1600 volts) for short periods (0.1 to 10 milliseconds) as opposed to cell inactivation due to membrane breakdown under the influence of strong fields. In general, it is thought to function by virtue of the elevated trans-membrane potential difference which leads to membrane structural rearrangements such that aqueous pathways or pores occur, thereby facilitating mass transfer processes. The influence of electric field pulses can greatly enhance the molecular transport through cell membranes, for example the electro-uptake of high molecular weight molecules to which cell membranes would normally be impermeable. The most important application of electroporation is the direct transfer of DNA into recipient cells of different origin (electrotransformation), while electrorelease is the use of strong electric field pulses to induce the release of cell ingredients e.g. to obtain intracellular proteins. The electroporative transfer of molecules (other than DNA), into recipient cells can also be achieved by electric fields. Examples of such molecules include proteins, antibodies, drugs, mutagens, and substances to which the cell membrane is poorly permeable or non-permeable.

The use of the technique in biotechnology has also been considered. Velizarov et al (1999) (1) stated that electroporation might also be applied to improve substrate utilisation efficiency in some biotechnological processes. An example given is the use of AC electric pulses of 0.25 kV for 10 ms to treat a yeast strain to enhance cellobiose utilisation and conversion to ethanol. In McCabe et al (1995) ethanol production was found to increase by almost 40% above that found in fermentations containing non-treated cells (2). In these examples, electroporation is achieved using AC fields.

Another general area of interest involves the effects of electric fields on cell growth and metabolite production. In this topic area, stimulation of cell proliferation by direct current has been studied. Generally, these have involved the transfer of chemicals produced by the electrode reaction to the cell enzymes, e.g. oxygen, hydrogen, ferrous ion, co-enzymes. An example is the use of electrolytically generated hydrogen as an electron donor for the hydrogenase enzyme to catalyse the reduction of precious metal ions to the metallic element (3).

The influence of pulsed electromagnetically induced currents, using devices such as pairs of Helmholtz coils, on processes in biotechnology has also been studied. Hones et al (1998) refer to an anaerobic bacterium expressing the enzyme nitrate reductase, which returns nitrogen to the atmosphere by denitrification. A low frequency alternating field induction in the mTorr range was used to give evidence of the possibility of accelerating cellular reactions. The authors state that the possible consequences for such fermentations are either the significant increase of biomass yield or an increase in the rate of fermentation. It was concluded that the pulsed electric field accelerates the cell division process, not the turnover number of the nitrate reductase.

The present inventors have now surprisingly found that when a DC electrical field is applied to a reaction mixture, biotransformation reactions can be increased under the influence of the electrical field when the reaction mixture is maintained or disposed separately from the means used to apply the field, such that it is not brought into contact therewith.

Therefore, according to a first aspect of the present invention, there is provided a process for increasing the rate of biocatalysis reactions, which comprises applying a direct current electric field to a reaction mixture, wherein the reaction mixture and the means to deliver said electric field are separated such that the reaction mixture does not come into contact with said electric field delivery means. The electrical field may be applied using techniques that are well known to the skilled practitioner, such as by the use of electrodes or the like in an electrochemical reactor. Advantageously, the enhancement in the rate of bioprocesses occurring in the reaction mixture results in increased turnover frequency (number of converted molecules per unit of time), decreased residence time (ratio of reactor volume to feed rate) and increased space-time yield (mass of product synthesised per reactor volume and time). Although the mechanistic pathways by which the enhancement effect is realised are currently unknown, because the electrodes are separated from the bioreaction mixture, no direct electron transport reactions can occur. Accordingly, the beneficial effect on bioreaction rate can only result from the influence of the DC electrical field. As set out more fully in the accompanying examples, the effect has been demonstrated to provide consistent rate improvements in specific biocatalysis reactions.

Thus, when the reaction mixture, which may include biological organisms, such as microorganisms, are physically protected from the area surrounding the electrodes and subsequent contact with the electrodes the effect is stimulatory.

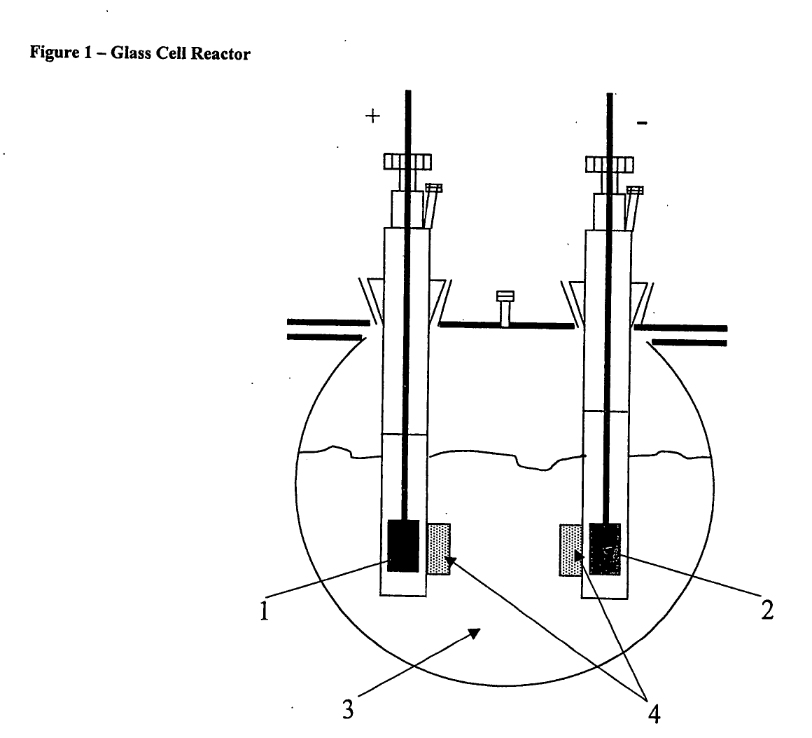

A number of methods will be apparent to the skilled practitioner for maintaining the electrodes separate from the reaction mixture and yet which can transmit or apply the electrical field to the reaction mixture. For example, the electrodes may be provided in a glass or other suitable container within the reaction mixture. In one embodiment, as identified in FIG. 1 each electrode is maintained in a glass tube having a porous window and containing an inert electrolyte, and which allows the passage of current to the bioreaction mixture but prevents any biomass in the mixture from contacting the electrodes.

Advantageously, the present invention can also be utilised in combination with modified electrodialysis to realise the continued effects of increased reaction rates with product separation and concentration. Significantly improved performance can be achieved in the process by utilising electrodialysis, particularly during extraction of products, for example from live biotransformation reactions, but also for batch treatment upon completion of the reaction.

The application of conventional electrodialysis methods, following completion of a batch biocatalysis reaction and removal of biomass by filtration of the mixture has been studied previously (5, 6). A variation of this technique is the use of bipolar membrane electrodialysis, which enables recovery of the product in a more purified form by separating the organic product from inorganic constituents (7), or preparation of an acid from a salt (8), again after completion of batch biotransformation reactions. Therefore, according to this aspect of the invention, the electrodes form part of an electochemical reactor, within which the reaction mixture is maintained, and which reactor includes a suitable electrodialysis membrane.

A further potential benefit provided by the use of an electrodialysis membrane such as a bipolar membrane is the continuous extraction and concentration of charged ionic organic products from live biocatalysis reactions. The approach would avoid the need to kill organisms on a routine basis, as required with an intermittent operational regime, and would provide the opportunity to maintain biocatalysis reactions under optimum pH conditions on a continuous operational basis. Reduced process costs would result from higher throughput rates and lower capital requirements, as well as lower consumption of chemicals and biocatalysts/biomass. Since the product would be maintained at a low concentration in the bioreaction mixture, any negative feedback inhibition of the biocatalysis process (e.g. observed in lactic acid fermentation reactions) would be avoided. In addition, the product would be recovered at much higher concentrations using electrodialysis membranes than could be produced directly in the biocatalysis reaction mixture which would improve the efficient isolation of pure product.

A number of difficulties must be overcome in order to improve the efficiency and cost effectiveness of product recovery by bipolar membrane electrodialysis, either from batch or continuous systems. A large number of ionic organic products are negatively charged at the pH levels necessary for biocatalysis reactions, e.g. carboxylates, and can be extracted from a reaction mixture through anion selective membranes. However, there are normally other components in the reaction mixture, which are also negatively charged, and will tend to move through the membrane, competing with the product anions. This has three major disadvantages. Firstly, the current efficiency for product recovery is reduced as a result of competitive anion transfer and the power requirement costs are significantly increased. Secondly, the anionic components of standard buffers, which are normally used in biocatalysis reactions, are transported from the reaction mixture and, as a result, the pH cannot be precisely controlled within the narrow limits necessary for efficient biocatalysis. Thirdly, the product stream is contaminated by the other anions transported, which can affect the final product recovery stage.

Thus, the anion selective membranes, as indicated more fully in the examples below, in addition to separating the reaction mixture from the electrodes, can also serve to transport organic acid anions through anion selective membranes from the bioreaction mixture through to a product stream.

In a preferred embodiment of the invention there is incorporated a cationic buffer system into the reaction mixture, in place of standard anionic buffer systems. This advantageously results in a substantial increase in the efficiency of the product removal process. In addition, by preventing the loss of buffer, the pH can be controlled automatically by adjusting the applied DC current, reducing the need for pH control by the addition of chemicals. It was observed that back-diffusion of product (e.g. lactic acid) was observed when the current was switched off. Therefore, at least a small residual current should be maintained in cases where a significant period without applied current would allow back-diffusion to occur. A typical cationic buffer system is “bis-Tris” Bis(2-hydroxyethyl)-imino-tris(hydroxymethyl)methane.

A further feature of the invention, which is enabled by the use of a cationic buffer system, is therefore the development of pH control without the addition of chemicals, by adjustment of the applied DC current. The pH control can be accomplished preferably by for example a computer-controlled current regulation system.

As the biocatalysis reaction proceeds, the pH falls as a result of the production of acidic product (e.g. benzoic acid, lactic acid etc.). In a batch process without continuous separation, addition of base is necessary throughout to maintain the pH of the reaction mixture within the range necessary. Continuous membrane extraction of the organic anion enables the replacement and neutralisation of the acid product by hydroxyl ion produced in the bipolar membrane by water splitting. However, if a standard anionic buffer system is being used in the separation system, the buffer is progressively displaced and pH control cannot be maintained. This problem has been overcome through the incorporation of a cationic buffer system which enables the pH to be controlled by the level of applied DC current through the membrane stack.

For each product anion transferred through the anion selective membrane from the reaction mixture to the product stream, an hydroxyl ion is produced by the bipolar membrane due to the splitting of water, which neutralises the acid produced by the biocatalysis process. Since the transfer of organic product anion out of the reaction mixture is quantitative, due to the presence of the cationic buffer, no buffer is displaced and the system is in balance. Example 6 and 8 are abiotic experiments in which the product benzoic acid (example 6) or lactic acid (example 8) is added to the reaction mixture to simulate its production in a fermentation process. This demonstrates this effect by matching the applied current to the addition rate of benzoic acid or lactic acid, and indicates that the product (benzoic acid or lactic acid) migration rate is linearly dependent on the applied current density.

A general advantage of electrochemical processes is therefore that automatic control can be readily effected, since it is possible to control the reaction rate by adjustment of the applied current. An aspect of this invention is the development of automatic pH control of biotransformation reaction mixtures, as part of continuous product recovery by modified electrodialysis.

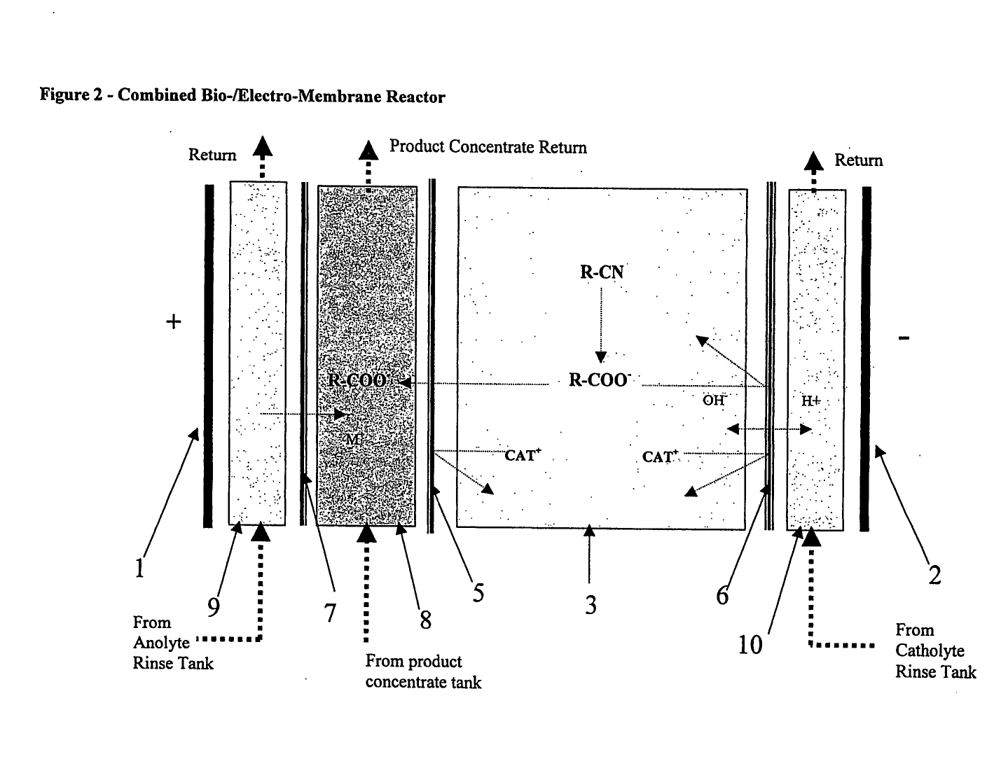

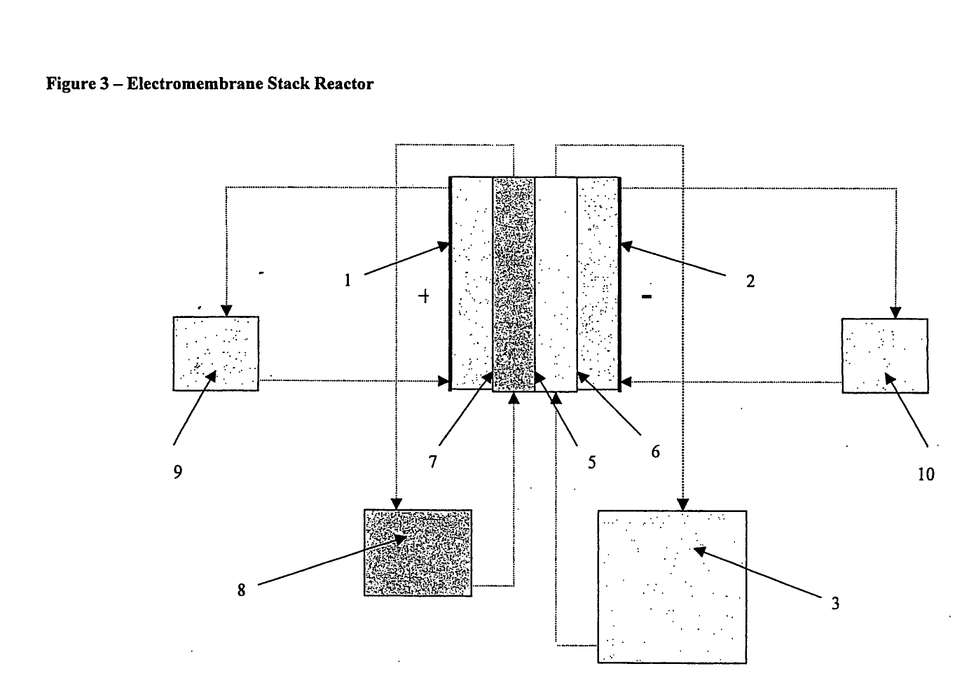

A useful, novel aspect of the present invention concerns the combination of the bio-reactor with the electro-membrane separation/concentration into a single integrated system, since the advantages of both the DC enhancement effect reaction rate and the improved electrodialysis product recovery system can be realised. The bio-reactor can be situated within an electrodialysis cell (as shown in FIG. 2) or the biomass can be contained mainly in a separate bio-reactor and re-circulated to an electrodialysis stack (as shown in FIG. 3). The current literature on the integration of electrodialysis processes with biocatalysis does not describe any interaction between the electric field and the biological material.

The improvements across the electrodialysis process, enabled by aspects of this invention, will provide more efficient, continuous operation of biocatalysis reactions. The solutions offered by the invention advantageously will allow bipolar electrodialysis to be applied to continuous extraction of ionic organic products from live biocatalysis reaction mixtures thus preventing competitive anion transfer resulting in the substantial increase in the current efficiency of the product separation process. In addition, the invention will enable control of the pH within the narrow optimal range essential for efficient operation of each specific biocatalysis reaction. Manual adjustment of the current supply has been suggested by prior workers as a method of controlling pH in lactic acid production by fermentation (9). However, imbalances in the system due to the transport of inorganic components in the fermentation broth were not taken into account. The improvements proposed will allow precise continuous control of pH. This may be achieved automatically by computer-controlled feedback loop to adjust the level of applied DC current and will obviate the need for pH control by chemical additions. Furthermore, the product concentrations will be maintained at low levels in the biocatalysis reaction mixtures, thus greatly reducing any negative feedback inhibition of catalyst activity. In addition to enabling continuous operation, the approach will also achieve substantial improvements in the efficiency of product isolation and concentration from batch processes.

The improvements in product separation/concentration offered by the invention can be applied to a broad spectrum of biocatalysis processes which involve the production of acidic organic compounds which are dissociated into negatively charged anions within the pH range necessary for high enzymatic activity. The approach has been applied to two specific reactions as set out more fully below, which are examples of two generic reaction types. Firstly, a whole cell single enzyme biotransformation, i.e. the conversion of benzonitrile to benzoic acid by Rhodococcus rhodochrous LL100-21. Secondly, a whole cell multi-enzyme step fermentation, i.e. lactic acid production from glucose by Lactobacillus rhamnosus NCIMB 6375.

The specific arrangements of the membranes according to the preferred aspects of the invention are provided in FIGS. 2 to 4. The bio-reaction mixture is contained between an anion-selective membrane on the anode-facing side and a bipolar membrane on the cathode facing side. When a standard buffer containing anionic components (e.g. borate) is present in the biocatalysis reaction mixture, competition is observed between the transport of the buffer anion and the organic acid anion. Even when small concentrations of anionic buffer are included, the current efficiency for product transfer is relatively low, e.g. the average current efficiency for benzoate transport is only 50% in the presence of 0.045 M boric acid. Because of this process inefficiency, substantially higher energy is consumed in achieving the desired rate of removal of the organic product. However, when a cationic buffer system (e.g. bis-Tris) is incorporated into the reaction mixture, so that there are no anions present other than the organic product, the applied current was fully utilised in the transport of organic product through the membrane. In example 7 a current efficiency of 75% was observed. The example is an abiotic experiment in which the addition of benzoic acid to the reaction mixture simulates its production during a fermentation process. TRIS buffer was used for the simulated bio-reaction stream. The competitive charge transport by migration of hydroxide produced at the bipolar membrane decreases the current efficiency, however, this can be reduced by higher organic product concentrations. The low solubility of benzoic acid in water prevents its use for an example to prove this point. In example 8, the current efficiency observed was close to 100%, significantly reducing the energy consumption and also increasing the rate of product separation. The example is an abiotic experiment in which the addition of lactic acid to the reaction mixture simulates its production during a fermentation process. Also, the presence of the bipolar membrane prevents the transport of the cationic buffer (bis-Tris in this case) towards the cathode, so that the buffer is retained within the bio-reaction mixture. As a result, the pH can be closely maintained without the consumption of expensive chemicals.

A common problem encountered when using membrane systems with microorganisms is membrane fouling. Regular membrane cleaning cycles can be introduced to the process to maintain the efficiency of the membrane operation (9).

However, the present invention has solved this problem by the immobilisation of microorganisms used which advantageously prevents fouling of membranes. The present inventors have found that membrane fouling is therefore either prevented or at least greatly reduced by using immobilised microorganisms, for example yeasts or bacteria. This effect was observed during the experiment given in Example 4, where the immobilisation medium was incorporated into a combined bio/electrodialysis reactor in order to benefit from the effects of both the DC field enhancement and reduced membrane fouling.

However, the volume of the electrodialysis stack reactor required to carry out this procedure in a combined bio/electrodialysis reactor would be very large. An alternative approach may be to contain the immobilisation media incorporating the microorganisms in a separate bioreactor of a standard design (continuously stirred, fluidised bed, packed bed etc.). However, no DC enhancement effect would be obtained in that case, since the microorganisms would not be exposed to the field. Therefore, a second DC field would be required, which may be applied to the bioreactor containing the immobilised microorganism, in order to obtain the DC enhancement effect in addition to product recovery/concentration. The second system would incorporate two bipolar membranes either side of the reactor to enable the field effects to be achieved without separation (as in Example 3). A relatively small area of bipolar membrane would be necessary in the bio-reactor to achieve the enhancement effect, while the relatively high membrane area required for product separation would be provided in a separate membrane stack.

The biocatalysis reaction according to the invention may comprise any of a single enzyme biotransformation reaction, a fermentation process, or a reaction catalysed by an isolated enzyme system. The reactions may be carried out with the assistance of growing microbial cultures, vesting microbial cultures or immobilised cultures of bacteria, fungi or yeasts.

The present invention will now be described by reference to the following examples and accompanying drawings wherein;

FIG. 1: is an illustration of a glass reactor consisting of a round-bottomed glass vessel with flanged lid. The Bio-Reaction Chamber (3) includes a spherical reaction vessel with total capacity of 250 ml. The Anode (1) and the Cathode (2) are housed in glass tubes, inserted through the flanged lid and contained in inert electrolyte. The electrodes are made of platinised titanium, with a rectangular surface of 2 cm×1.5 cm. The tubes are immersed in the biotransformation reaction mixture contained within the flask. The reaction mixture is separated from the anode and cathode compartments by Porous Glass Windows (4). The separators allow the passage of current, but prevent the biomass from contacting the electrodes. No product separation was involved.

FIG. 2: is an illustration of a combined Bio-/Electro-Membrane Reaction used in the present invention.

This reactor design enables the Bio-reactor chamber to be integrated within a modified electrodialysis stack containing ion selective and biopolar membranes, so that the effects of biotransformation reaction rate enhancement and product separation can be combined.

The reactor contains a Bio-Reaction Chamber (3) made of Perspex with a 1.4 L working capacity, containing the entire volume of the biotransformation reaction mixture. A magnetic stirrer continuously mixes the medium containing the cell mass in the central chamber, but there is no circulation of this mixture to an external vessel. Bolted onto one side are two further chambers and the Anode (1) (platinised titanium). An Anion Selective Membrane (5) (Neosepta ACM) is situated between the Bio-Reaction Chamber and the Product Concentrate Chamber (8). The product chamber is separated from the Anolyte Chamber (9) by a cation selective membrane (7) (DuPont Nafion 450). Bolted on the other side of the Bio-Reaction Chamber are the Catholyte Chamber (10) and the Cathode (2) (stainless steel). A Bipolar Membrane (6) (Tokuyama Co. Ltd. BPM 1) separates the Bio-Reaction Chamber and the catholyte chamber. Chambers (8), (9) and (10) are much thinner than chamber (3) and are fitted with inlets and outlets to enable the solutions to be circulated to separate external reservoirs at the speed of 20 ml/min. The electrodes of the reactor have a rectangular surface of 10×10 cm.

FIG. 3: is an illustration of an Electro-Membrane Stack Reactor used in accordance with the present invention.

The reactor design enables a generic, industrial electro-membrane stack system to be used for the enhancement of bio-transformation reaction rate and separation of product. The reaction mixture, containing biomass, is re-circulated from a separate bio-reactor, through the Bio-Reaction Chamber of the membrane stack reactor.

The stack contains four chambers fabricated from HDPE with dimensions 3×160×230 mm. The active area of each membrane exposed by the frames was 120×160 mm. A polymer mesh (HDPE) is provided in each frame to provide membrane support and good hydrodynamic flow. The Bio-Reaction Chamber dimensions are identical to the other chambers. All solutions are re-circulated to external vessels. In the case of the reaction mixture, the biomass is in suspension and therefore passes through the electro-membrane stack and is exposed to the DC field for part of the time.

The two end plates incorporate recessed electrodes. The anode (tantalum/iridium oxide coated titanium) is in contact with a bead of Silicone Sealant which fills an annular groove machined in the electrode recess PVC plate. The cathode (titanium) is similarly sealed into the PVC support plate. The electrodes have a rectangular surface of 120×160 mm.

The arrangement of electrodes and membranes is identical to that described in the Combined Bio-/Electro-Membrane Reactor (component numbering is the same).

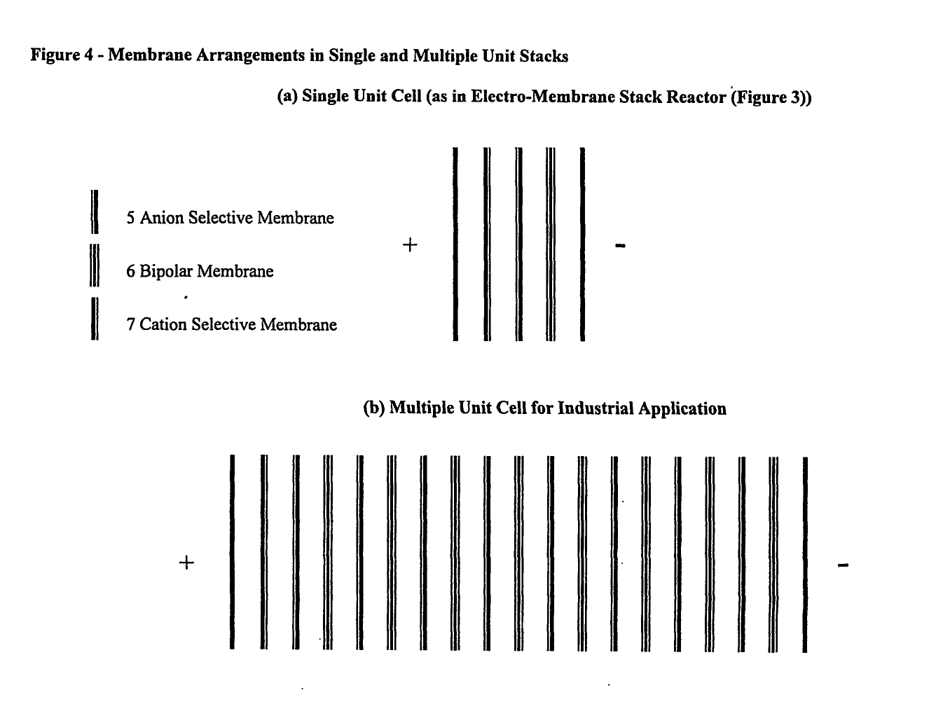

FIG. 4: is an illustration of Membrane Arrangements used in Single and Multiple Unit Stacks.

The arrangement of membranes used in the Electro-Membrane Stack Reactor is given in FIG. 4. In FIG. 4(a) the system contains a single unit of one bipolar membrane and one anion selective membrane, with a single cation selective membrane adjacent to the anolyte compartment. FIG. 4(b) illustrates the arrangement used in a multiple unit cell for industrial application.

### EXAMPLE 1

This example demonstrates that the biotransformation enhancement effect is observed in a simple glass reactor, in which the bio-reaction mixture is prevented from contacting the electrodes by the inclusion of porous separators.

Starter cultures of Rhodococcus rhodococcus (LL100-21) were grown at 30° C. in 50 ml of a liquid medium in an orbital shaker (160 rpm) until the late exponential phase of growth. The medium contained: TRIS Borate Buffer (0.045 M H₃BO₃, 0.33 M TRIS—Tris-(hydroxymethyl)aminomethane) (TBB), 0.5 mM KH₂PO₄, 8.6 mM NaCl, 0.01 M CaCl₂, 0.1 M MgSO₄ and 1 ml/L of trace elements (Beauchop, 1960). During the reaction, benzonitrile provided the sole source of carbon and nitrogen. To produce cells for biotransformation a 4% w/v inoculum of starter cells was used in a final volume of 500 ml and the culture was grown aerobically at 30° C. in an orbital shaker (160 rpm) for 36 hours. Cells were finally harvested in early/mid-exponential phase of growth by centrifugation for 15 min at 13000 g. The cells were washed in TBB three times and then re-suspended in 200 ml TBB into the Glass Cell Reactor. Each Electrode chamber contained 15 ml TBB. Bacteria were placed directly into M and 10 mM benzonitrile was added. A current of 16.6 A/m² DC was applied for a period of 260 minutes. In the presence of the constant electric current the average production rate was 0.027 mmol/min/g dcw (dry cell weight).

The experiment was repeated under the same conditions without applying a constant electric current to the cells in the glass cell reactor. The average production rate in the absence of electric current was 0.020 mmol/min/g dcw.

This example shows that electric current increases the biotransformation rate by 35%.

### EXAMPLE 2

This example demonstrates that the biotransformation enhancement effect is observed in the combined Bio-Electro-Membrane Reactor, and that product separation/concentration can be achieved simultaneously. Starter cultures of bacteria (50 ml) were prepared as described in example 1. A 4% w/v inoculum of starter cells was used in a final volume of 500 ml and the culture was grown at 30° C. for 36 hours. Cells were finally harvested in mid/late-exponential phase of growth by centrifugation for 15 min at 13000 g. The cells were washed in TBB three times and then re-suspended in 1.4 L TBB into the Combined Bio-/Electro-Membrane Reactor. The pH of the product concentrate was maintained at pH 8 through addition of 4 N NaOH by means of a pH control system comprising a DIN rail mounted pH controller, a variable pump, and a pH electrode. 500 ml of TBB was used as electrolyte for chambers (8), (9) and (10).

10 mM benzonitrile was added to the reaction mixture to initiate the biotransformation. DC current was switched on and set to deliver a constant current of 16.6 A/m². In the presence of the constant electric current the average production rate was 0.044 mmol/min/g dcw. Benzoic acid migrated into the Product Concentrate Chamber with a migration rate of 0.02 mmol/min.

The experiment was repeated under the same conditions without applying a constant electric current to the cells in the electrokinetic reactor. The average production rate in the absence of electric current was 0.031 mmol/min/g dcw.

This example shows that electric current increases the biotransformation rate by 42% and that concentrated benzoic acid can be recovered by electrodialysis.

### EXAMPLE 3

This experiment was conducted in order to demonstrate that the biotransformation enhancement effect did not result from the product separation/concentration process, by replacing the anion selective membrane with a bipolar membrane in the Combined Bio-Electro-Membrane Reactor. The cell mass of Rhodococcus rhodococcus LL100-21 was grown and harvested in the same way as in example 2. The cells were re-suspended in 1.4 L TBB and added to the Combined Bio-/Electro-Membrane Reactor. However, this reactor was modified by replacing the anion selective membrane (used in Example 2) between the Bio-Reaction Chamber and the Product Concentrate Chamber by a bipolar membrane (Tokuyama Neosepta BP-1), therefore no benzoic acid product was transported out of the reaction mixture. 10 mM of benzonitrile was added to the cells and DC current was switched on (16.6 A/m²). In the presence of the constant electric current the average production rate was 0.023 mmol/min/g dcw. The experiment was repeated under the same conditions without applying a constant electric current to the cells in the reactor. The average production rate in the absence of electric current was 0.018 mmol/min/g dcw. This example shows that electric current stimulates bacterial metabolism increasing the biotransformation rate by 28%.

### EXAMPLE 4

This example demonstrates that the biotransformation enhancement effect is observed using immobilised biomass in the combined Bio-Electro-Membrane Reactor, and that product separation/concentration can be achieved simultaneously. Starter cultures of bacteria (50 ml) were prepared as described in example 1. A 4% w/v inoculum of starter cells was used in a final volume of 500 ml and the culture was grown at 30° C. Once bacterial cultures had reached the late exponential phase of growth they were harvested by centrifugation at 13000 g for 15 min. The supernatant was discarded and bacteria were re-suspended in TBB to a final density of 5 g/L. The cell concentrate was then mixed with an equal volume of 4% alginate. The dense suspension was added drop-wise through a hypodermic needle into 1 L of 0.25 M CaCl₂ in order to allow the solidification of the alginate into regular spheres. These alginate beads containing the bacteria were harvested by use of a sieve, washed once with TBB and suspended in 1.4 L TBB in the Bio-Reaction Chamber of the Combined Bio-/Electro-Membrane Reactor illustrated in FIG. 2, using the standard membrane arrangement, as in Example 2. 10 mM of benzonitrile was added to the cells and DC current was switched on (16.6 A/M²). In the presence of the constant electric current the average production rate was 0.042 mmol/min/g dcw. Benzoic acid migrated into the Product Concentrate Chamber MA with a migration rate of 0.02 mmol/min. The experiment was repeated under the same conditions without applying a constant electric current to the cells in the reactor. The average production rate in the absence of electric current was 0.032 mmol/min/g dcw. This example shows that electric current increases the biotransformation rate of the immobilised microorganisms by 31%.

### EXAMPLE 5

This example is included to demonstrate that the biotransformation enhancement effect is observed in the Electro-Membrane Stack Reactor, in spite of the fact that the biomass is exposed to the influence of the applied DC field for part of the reaction time only, due to circulation from an external bioreactor. Starter cultures of bacteria (50 ml) were prepared as described in example 1. A 4% w/v inoculum of starter cells was used in a final volume of 500 ml and the culture was grown at 30° C. for 36 hours. Cells were finally harvested in mid/late-exponential phase of growth by centrifugation for 15 min at 13000 g. The cells were washed in TBB three times and then re-suspended in 0.5 L TBB and transferred into a glass flask fermenter of 0.6 litre in capacity, which was asceptically connected to the Bio-Reaction Chamber of the Electro-Membrane Stack Reactor. 500 ml of TBB was used as electrolyte for the Anolyte, Catholyte and Product Concentrate Chambers. The pH of the Product Concentrate Chamber was maintained at pH 8 by means of a pH control system described in Example 2. 10 mM of benzonitrile was added to the cells and biotransformation was carried out by Rhodococcus rhodococcus LL100-21 in the absence of electric current in the reactor for 210 minutes. The biotransformation rate was 0.030 mmol/min/g dcw. After 210 min 10 mM of benzonitrile was added again and DC current was switched on (16.6 A/M²). In the presence of the constant electric current the average production rate was 0.044 mmol/min/g dcw. Benzoic acid migrated into the Product Concentrate Chamber with a migration rate of 0.02 mmol/min. This example shows that electriccurrent increases the biotransformation rate by 47% and that concurrent separation and concentration of benzoic acid can be achieved with this electro-membrane stack.

### EXAMPLE 6

This example, in which the Electro-Membrane Stack Reactor was operated in abiotic mode, demonstrates that pH can be precisely controlled by the variation of applied current. One litre of TBB was circulated through each the Anolyte and Catholyte Chamber and one litre of TBB containing 11 mmol benzoic acid was circulated through the Product Concentrate Chamber. One litre of TBB containing 5 mmol benzoic acid was circulated through the Bio-reaction chamber. DC current was supplied to the electrodialysis stack. A concentrated solution of benzoic acid was added continuously to the solution circulated through the Bio-Reaction Chamber with varying addition rates. The current to be applied was changed manually according to the benzoic acid addition rate to maintain the pH in the Bio-reaction chamber at 8. Benzoic acid was first added with an addition rate of 0.025 mmol/min. The supplied current was 0.09 A. After 4% hours the addition rate was decreased to 0.012 mmol/min and the current was changed to 0.04 A. Half an hour afterwards benzoic acid was added with a rate of 0.041 mmol/min and the current was changed to 0.15 A. After 5½ hours operation 20 mmol of benzoic acid (82% of the total benzoic acid) were recovered in the Product Concentrate stream. 4 mmol of benzoic acid remained in the Bio-Reaction stream (16% of the total benzoic acid). The average current efficiency was 50%. During the experiment the pH in the Bio-Reaction stream stayed between 8.0 and 8.1.

This example shows that the pH in the reaction mixture can be controlled by adjustment of the applied current and that the migration rate of the product (benzoic acid in this case) is linearly dependent on the applied current density.

### EXAMPLE 7

This example, in which the Electro-Membrane Stack Reactor was operated in abiotic mode, demonstrates that higher current efficiencies can be achieved by avoiding other anions than the product (benzoate in this case) in the Bio-reaction stream and that pH stability can be obtained, due to the presence of TRIS buffer, Tris-(hydroxymethyl)aminomethane. A half litre of a 0.1 M Na₂SO₄ solution was circulated through each the Anolyte and Catholyte Chambers. A half litre of TBB was circulated through the Product Concentrate Chamber. A half litre of a 0.5 M Trizma base solution containing 5 mmol benzoic acid was circulated through the Bio-reaction chamber. DC current (0.15 A) was supplied to the electrodialysis stack. A concentrated solution of benzoic acid was added continuously to the solution circulated through the Bio-Reaction Chamber with an addition rate of 0.07 mmol/min. After 5% hours operation 22 mmol of benzoic acid (78% of the total benzoic acid) were recovered in the Product Concentrate stream. 6 mmol of lactic acid remained in the Bio-Reaction stream (21% of the total benzoic acid). The average current efficiency was 75%.

During the experiment the pH in the Bio-Reaction stream stayed between 8.3 and 8.4. This example shows that avoiding other anions than the product (benzoate in this case) in the bio-reaction stream increases the current efficiency. At the same time, because of the use of TRIS buffer, the pH remains stable in the biotransformation reaction medium.

### EXAMPLE 8

This example, in which the Electro-Membrane Stack Reactor was operated in abiotic mode, demonstrates that high current efficiencies can be achieved for product separation/concentration and that pH stability can be obtained, due to the presence of cationic buffer. It also demonstrates that higher current efficiencies can be achieved by higher product concentrations, which minimise competitive charge transport by hydroxide migration. Lactic acid was used as a model product instead of benzoic acid, because the low solubility of benzoic acid in water prevents its use in higher concentrations. One litre of 0.1 M sulphuric acid was circulated through the Anolyte and the Catholyte Chambers, and 1.1 litre of a 1.6 M lactic acid solution was circulated through the Product Concentrate Chamber. A solution (0.5 litre) of 0.05 M bis-Tris buffer containing 0.1 mol lactic acid was circulated through the Bio-Reaction Chamber. DC current was supplied to the stack. Lactic acid (85%) was added continuously to the solution circulated through the Bio-Reaction Chamber with varying addition rates. The current to be applied was changed manually according to the lactic acid addition rate to maintain the pH at 6. Lactic acid was first added with an addition rate of 2.4 mmol/min for 4 hours. The supplied current was 3.5 A. After 4 hours the addition rate was increased to 3.6 mmol/min and the current was changed to 5.2 A. 1½ hours afterwards lactic acid was added with a rate of 4.8 mmol/min and the current was changed to 6.9 A. After 6½ hours operation 2.95 mol of lactic acid (97% of the total lactic acid) were recovered in the Product Concentrate stream. 0.03 mol of lactic acid remained in the bio-reaction stream (1% of the total lactic acid). The average current efficiency was 100%. During the experiment the pH in the simulated reaction mixture was successfully maintained between 5.9 and 6.3. This example shows that the product (lactic acid) can be recovered with this electro-membrane stack design and that the use of a cationic buffer and higher product concentration (lactic acid in this case) in the Bio-Reaction stream increases the current efficiency.

Therefore, novel effects of the invention have been demonstrated in a laboratory glass reactor, given in FIG. 1. The experimental details and results of which are given in Example 1. This example demonstrated that the application of a DC electric field increased the biotransformation rate by 35%.

The novel effect has also been reproduced in a laboratory-scale modular laboratory reactor design, given in FIG. 2. This type of electro-membrane electrochemical reactor design is a suitable model design for scale-up to industrial-scale. The experimental details are given in Examples 2, 3 and 4.

In example 2, the product (benzoic acid) was also transferred continuously from the biotransformation reaction mixture by the DC field through an anion selective membrane into a product stream. In Example 3, the anion membrane was replaced by a bipoloar membrane, so that no transfer of product was possible. In Example 4, separation of product (as carried out in Example 2) was combined with immobilisation of the bacterial cells on alginate beads. These examples demonstrated that the application of a DC electric field increased the biotransformation rates in Examples 2, 3 and 4 by 42%, 28% and 31% respectively.

The novel effect has also been reproduced in a laboratory-scale electro-membrane stack, given in FIG. 3. The design of this reactor was suitable for direct scale-up to industrial plant. The experimental details are given in Example 5. The system was operated with continuous separation of benzoic acid using an anion exchange membrane. In this example, the DC field enhanced the biotransformation rate by 47%. In this case, the bacteria were recycled from an external reservoir through the electrodialysis stack. Therefore, the result confirms that the enhancement effect can be maintained when the electrochemical reactor is situated outside the main bioreactor.

Unlike electroporation, which employs AC fields, the new effect is achieved through the application of DC fields. Also, prior work on increased cell growth and metabolite production has involved pulsed electromagnetically induced AC currents.

### REFERENCES

1) S. Velizarov; Electro- and Magnetobiology, 18(2), 185-212 (1999).

2) A. McCabe, N. Barron, L. McHale and A. P. McHale; Biotechnol. Tech. 9, 133-136 (1995).

3) P. Yong, J. P. G. Farr, I. R. Harris and L. Macaskie; Biotechnology Letters 24, 205-212 (2002).

4) I. Hönes, A. Pospischil, H. Berg; Bioelectrochemistry and Bioenergetics 44, 275-277 (1998).

5) F. Miao; U.S. Pat. No. 5,681,728 (Oct. 28, 1997).

6) R. Cabacang, M. Kishimoto, K. Suga; Biotechnology for Sustainable Utilisation of Biological Resources in the Tropics 14, 226-235 (2000).

7) J. D. Genders, R. Gopal, D. M. Hartsough, P. M. Kendall, W. J. Long, D. J. Mazur, G. D. Zappi; U.S. Pat. No. 6,187,570 (Feb. 13, 2001).

8) O. Favre-Bulle, J. Pierrard, C. David, P. Morel, D. Horbez; U.S. Pat. No. 6,180,359 (Jan. 30, 2001).

9) Y. Nomura, M. Iwahara and M. Hongo; Biotechnology and Bioengineering 30, 788-793 (1987).

1. A process for increasing a rate of a biocatalysis reaction, which comprises,
applying a direct current (DC) electric field to a reaction mixture, wherein the reaction mixture and electrodes used to apply said electric field are separated such that the reaction mixture does not come into contact with said electrodes.

2. A process according to claim 1, wherein said electric field is applied for a sufficient time to stimulate the biocatalysis reaction in the reaction mixture.

3. A process according to claim 1, wherein said reaction mixture and said electrodes are separated by a separation membrane.

4. A process according to claim 3, wherein said separation membrane is any of an ion exchange membrane or a microporous membrane.

5. A process according to claim 4, wherein said separation membrane is a bipolar ion exchange membrane.

6. A process according to claim 1, wherein said electrodes form part of an electrochemical reactor.

7. A process according to claim 6, wherein said electrochemical reactor forms part of an electrodialysis stack, wherein charged organic products in the reaction mixture can be removed by electrodialysis.

8. A process according to claim 1, wherein said reaction mixture is contained between a bipolar membrane on a cathode-facing side and an anion selective membrane on an anode-facing side of said reaction mixture.

9. A process according to claim 1, wherein the reaction mixture comprises a cationic buffer system, with an organic product forming an anionic component.

10. A process according to claim 9, wherein the DC electric field applied is adjusted to control pH of the reaction mixture.

11. A process according to claim 10, wherein adjustment to the DC electric field is automatically controlled under control of a computer program.

12. A process according to claim 7, wherein the biocatalysis reaction and the electrodialysis are operated in separate, but linked, reactors, where the biocatalysis reaction mixture containing active biomass can be recirculated continuously to the electrochemical reactor.

13. A process according to claim 1, wherein the biocatalysis reaction is selected from a single enzyme biotransformation reaction, a fermentation process and a reaction catalyzed by an isolated enzyme system.

14. A process according to claim 1, wherein the reaction mixture comprises any of growing or resting microbial cultures.

15. A process according to claim 14, wherein said microbial cultures comprise immobilized cultures of yeast, bacteria or fungi.

16. A process according to claim 15, wherein said cultures are immobilized on surfaces or in pores of beads.

17. A process according to claim 8, wherein the reaction mixture comprises a cationic buffer system, with an organic product forming an anionic component.

18. A process according to claim 8, wherein the reaction mixture comprises immobilized microbial cultures of yeast, bacteria or fungi.

19. A process according to claim 8, wherein the reaction mixture comprises immobilized microbial cultures on surfaces or in pores of beads.

20. A process according to claim 9, wherein the reaction mixture comprises immobilized microbial cultures of yeast, bacteria or fungi on surfaces or in pores of beads.
